(19) United States
(12) Patent Application Publication
(10) Pub. No.: US 2019/0150438 A1
(43) Pub. Date: May 23, 2019

(54) CONFIGURATIONAL STEREOISOMER OF DIFETHIALONE, COMPOSITION AND RODENTICIDE BAIT COMPRISING SAME, AND METHOD FOR CONTROLLING TARGET RODENT PESTS

(71) Applicants: LIPHATECH, Pont-Du-Casse (FR); INSTITUT ENSEIGNEMENT SUPERIEUR ET RECHERCHE EN ALIMENTATION SANTE ANIMALE SCIENCES AGRNOMIQUES ET, Marcy L'Etoile (FR)

(72) Inventors: Hervé CARUEL, Moncaut (FR); Etienne BENOIT, Lyon (FR); Isabelle FOUREL, Miribel (FR); Virginie LATTARD, Lyon (FR)

(21) Appl. No.: 16/061,062

(22) PCT Filed: Dec. 6, 2016
(86) PCT No.: PCT/EP2016/079857

§ 371 Date: Jun. 11, 2018

(30) Foreign Application Priority Data
Dec. 11, 2015 (FR) 1562226

(51) Int. Cl. A01N 43/18; A01N 25/00; C07D 335/06

(52) U.S. Cl. CPC A01N 43/18; C07B 2200/07; C07D 335/06; A01N 25/004

(57) ABSTRACT

Disclosed is a dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations.

The invention relates to a configurational stereoisomer of difethialone as an isolated compound, to a composition and a rodenticidal bait comprising such a configurational stereoisomer and to a process for controlling target rodent pests. The invention thus relates to the technical field of controlling populations of target rodent pests.

It is known practice to use rodenticidal baits as poisons for target rodent pests. It is known from EP 2 090 164 that difethialone is a second-generation anticoagulant acting in a single dose. US 2005/181003 describes a rodenticidal bait in gel form comprising difethialone in a mass proportion of 25 ppm.

Such a bait is liable to be consumed by animals other than target rodent pests when it is made available to target rodent pests. It may be consumed directly (primary consumption) by domestic animals or pets. It may also be consumed accidentally by humans. Such consumption may result in poisoning, which may be lethal, of these domestic animals or pets or of humans.

In addition, a fraction of the difethialone of these rodenticidal baits may be ingested (secondary consumption) by animals—especially by birds—which prey on weakened rodent pests that have consumed such a rodenticidal bait, or by animals which carrion-feed on rodent pests that have died from having consumed such a rodenticidal bait. This secondary consumption is liable in the long term to result in the death of these predatory or carrion-feeding animals, which may be animals—especially birds—belonging to protected species.

The invention is thus directed towards overcoming these drawbacks by proposing a configurational stereoisomer of difethialone, a composition and a rodenticidal bait comprising such a configurational stereoisomer and a process for controlling target rodent pests, which are efficient for controlling populations of target rodents.

The invention is also directed towards proposing a configurational stereoisomer of difethialone, a composition and a rodenticidal bait comprising such a configurational stereoisomer and a process for controlling target rodent pests, the use of which is in accordance with the rules of good practice, especially with respect to the protection of birds, and in particular birds of prey.

The invention is also directed towards proposing a configurational stereoisomer of difethialone, a composition and a rodenticidal bait comprising such a configurational stereoisomer and a process for controlling target rodent pests, which do not require, in order to control a population of target rodent pests, the use of massive doses of rodenticidal agents and which are friendly towards the environment and the health of humans and non-target animals.

The invention is thus directed towards proposing a configurational stereoisomer of difethialone, a composition and a rodenticidal bait comprising such a configurational stereoisomer and a process for controlling target rodent pests, which is efficient with a reduced dose of rodenticidal agent.

The invention is also directed towards proposing a configurational stereoisomer of difethialone, a composition and a rodenticidal bait comprising such a configurational stereoisomer and a process for controlling target rodent pests, which are able to be used for controlling target rodent pests that are resistant to known rodenticidal baits.

The invention is thus directed towards proposing an alternative to known rodenticidal baits.

To do this, the invention relates to a dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations.

Throughout the text:
the term “difethialone” denotes the compound 3-(4′-bromobiphenyl-4-yl)-1-(4-hydroxythiocoumarin-3-yl)-1,2,3,4-tetrahydronaphthalene or 3-[3-[4-(4-bromophenyl)phenyl]-1-tetralinyl]-2-hydroxy-4-thiochromenone or 3-[3-(4′-bromo[1,1′-biphenyl]-4-yl)-1,2,3,4-tetrahydro-1-naphthalenyl]-4-hydroxy-2H-1-benzothiopyran-2-one of formula (I) below:

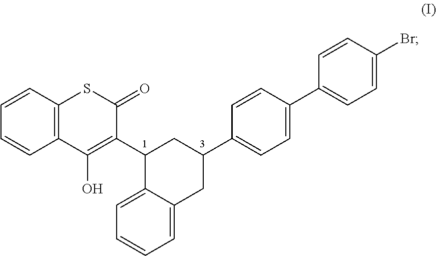 [SMILES: O=c1sc2ccccc2c(O)c1C1CC(c2ccc(-c3ccc(Br)cc3)cc2)Cc2ccccc21]

in which are indicated the numbers of carbons 1 and 3 of the 1,2,3,4-tetrahydronaphthalene group;
the term “stereoisomers” denotes isomers of the same semi-structural formula, but in which the relative position of the atoms differs in space. The term “configurational stereoisomers” denotes stereoisomers for which conversion from one to the other of these configurational stereoisomers requires the cleavage/reformation of an interatomic covalent bond. Thus, the term “configurational stereoisomers” denotes stereoisomers which are not conformational isomers (or “rotamers”, for which conversion from one to the other of the conformational isomers is accompanied only by rotation of a part of the molecule about the axis of a σ (sigma) bond formed by axial orbital overlap);
the term “hetero-stereoisomer” of difethialone denotes the configurational stereoisomer of difethialone of formula 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group of said hetero-stereoisomer have different absolute configurations (1R,3S and 1S,3R), said absolute configurations being determined according to the sequential priority rules and the Cahn-Ingold-Prelog (CIP) nomenclature;
the term “homo-stereoisomer” of difethialone denotes the configurational stereoisomer of difethialone of formula 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group of said homo-stereoisomer have the same absolute configuration (1R,3R and 1S,3S);
the term “amount” means a molar amount, a mass amount or a volume amount. The proportions are thus proportions of a molar amount relative to a molar amount, of a mass amount relative to a mass amount, or of a volume amount relative to a volume amount;
the term “substantially” indicates, in the usual manner, that a structural or functional characteristic should not be taken as marking an abrupt discontinuity, which would have no physical meaning, but covers not only this structure or this function, but also slight variations of this structure or of this function which produce, in the technical context under consideration, an effect of the same nature, or else of the same degree;
the expressions “high-pressure liquid chromatography” or “high-performance liquid chromatography” (HPLC) denote “HPLC” chromatography or “High Performance Liquid Chromatography”; and
the term “retention time” denotes the time, measured at the top of the peak in the chromatogram, for which a compound is retained on a chromatography column.

The invention thus relates to the dextrorotatory enantiomer of said hetero-stereoisomer of difethialone in isolated form. The invention relates to this dextrorotatory enantiomer of said hetero-stereoisomer of difethialone separated from the laevorotatory enantiomer of said hetero-stereoisomer of difethialone and separated from the enantiomers of said homo-stereoisomer of difethialone.

The inventors succeeded in separating the laevorotatory and dextrorotatory enantiomers of said hetero-stereoisomer of difethialone and the laevorotatory and dextrorotatory enantiomers of said homo-stereoisomer of difethialone by high-pressure liquid chromatography in isocratic mode and under particular conditions by using a chromatography column comprising a specific chiral stationary phase and in isolating the dextrorotatory enantiomer of said hetero-stereoisomer of difethialone. Specifically, it was not known at the date of the invention how to separate the laevorotatory enantiomer of said hetero-stereoisomer of difethialone and the dextrorotatory enantiomer of said hetero-stereoisomer of difethialone.

This separation is performed using a LUX® Cellulose-3 HPLC column (Phenomenex, Le Pecq, France) of dimensions 150×2 mm and comprising a chiral stationary phase constituted of porous particles of tris(4-methylbenzoate) cellulose, having a particle size of 3 μm and a porosity of 1000 Å. The mobile phase used is an eluent obtained by mixing acetonitrile (A) and water comprising formic acid in a volume proportion of 0.1% in the water (B) with an A/B volume ratio of 80/20. The flow rate of the mobile phase in the column is maintained at a value of 0.25 mL/minute and the separation is performed at a temperature of 23.2° C. The composition to be analysed is at a concentration of 1 μg of difethialone per millilitre in acetonitrile and the volume of composition injected onto the column is 1 μL. Detection may be performed by tandem mass spectrometry (MS/MS) at the outlet of the separating column. Detection and quantification may be performed by photometry or by spectrophotometry by adjusting the difethialone concentration and the injection volume for the purpose of obtaining optimum detection and by measuring the value of the area under the peak for each configurational stereoisomer.

Under these analytical conditions, the value of the retention time (t₃) for the dextrorotatory enantiomer of said hetero-stereoisomer of difethialone may vary depending on the operating conditions—especially on the column temperature conditions—and may be between 11.3 minutes and 11.8 minutes. The value of the retention time (t₂) for the laevorotatory enantiomer of said hetero-stereoisomer of difethialone may vary depending on the operating conditions—especially depending on the column temperature conditions—and may be between 9.0 minutes and 9.5 minutes, such that the dextrorotatory and laevorotatory enantiomers of said hetero-stereoisomer may be separated by high-pressure liquid chromatography on a chiral column.

The value of the retention time (t₁) for the laevorotatory enantiomer of said homo-stereoisomer of difethialone may vary depending on the operating conditions—especially on the column temperature conditions—and may be between 7.8 minutes and 8.2 minutes. The value of the retention time (t₄) for the dextrorotatory enantiomer of said homo-stereoisomer of difethialone may vary depending on the operating conditions—especially depending on the column temperature conditions—and may be between 14.0 minutes and 14.4 minutes, such that the dextrorotatory and laevorotatory enantiomers of said homo-stereoisomer of difethialone may be separated by high-pressure liquid chromatography on a chiral column.

Thus, under these conditions, the order of elution of the configurational stereoisomers of difethialone is such that t₁<t₂<t₃<t₄. The retention time values t₁, t₂, t₃ and t₄ are liable to vary, especially with the temperature of the chromatography column. However, under these chromatographic separation conditions, the order of elution of the configurational stereoisomers of difethialone remains unchanged.

The dextrorotatory enantiomer of said hetero-stereoisomer of difethialone isolated in pure form according to the invention, dissolved in methanol (CH₃OH) at a concentration of 0.65 g/L and placed in a quartz spectrophotometry cuvette, has a circular dichroism spectrum acquired at 25° C. with positive circular dichroism values between 210 nm and 250 nm and between 270 nm and 300 nm.

The dextrorotatory enantiomer of said hetero-stereoisomer of difethialone isolated in pure form according to the invention, dissolved in chloroform (CHCl₃), has a specific optical rotation [α]^{25° C.}₅₈₉ nm, measured at 25° C. and on the sodium D line (589 nm), having a mean value obtained on two series of ten measurements of +9.1°.

The dextrorotatory enantiomer of said hetero-stereoisomer of difethialone isolated in pure form according to the invention has, on proton magnetic resonance (¹H-NMR) spectroscopy at 500 MHz in CDCl₃, a multiplet with a chemical shift (δ) of between 5.2 ppm and 5.4 ppm—especially about 5.3 ppm—corresponding to the proton borne by carbon 1 of the 1,2,3,4-tetrahydronaphthalene group of said hetero-stereoisomer of difethialone.

Said hetero-stereoisomer of difethialone and said homo-stereoisomer of difethialone are distinguished by their proton NMR spectra. In the proton NMR spectrum acquired in CDCl₃, the chemical shift of the proton borne by carbon 1 of the 1,2,3,4-tetrahydronaphthalene group of said homo-stereoisomer of difethialone is between 4.9 ppm and 5.1 ppm.

The invention also relates to a composition comprising the dextrorotatory enantiomer of said hetero-stereoisomer of difethialone according to the invention, with the exclusion of a racemic mixture of dextrorotatory and laevorotatory enantiomers of said hetero-stereoisomer of difethialone.

The invention thus also relates to a composition comprising a dextrorotatory enantiomer of a configurational stereoisomer of difethialone, named hetero-stereoisomer, of formula 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations, with the exclusion of a mixture in which the dextrorotatory enantiomer and the laevorotatory enantiomer of said hetero-stereoisomer of difethialone are in equal amounts. In a composition according to the invention, the dextrorotatory enantiomer of said hetero-stereoisomer and the laevorotatory enantiomer of said hetero-stereoisomer of difethialone are in unequal (different) amounts.

Advantageously and according to the invention, said hetero-stereoisomer is predominantly in dextrorotatory enantiomer form in the composition. Advantageously, a composition according to the invention comprises difethialone stereoisomer predominantly in dextrorotatory enantiomer form.

Throughout the text, the term “said hetero-stereoisomer is predominantly in dextrorotatory enantiomer form” means that the (mass, molar or volume) amount of dextrorotatory enantiomer of said hetero-stereoisomer of difethialone is the major amount—greater than 50%—in all of said hetero-stereoisomer of difethialone present in the composition (in all its dextrorotatory and laevorotatory enantiomer forms), i.e. the (mass, molar or volume) amount of dextrorotatory enantiomer of said hetero-stereoisomer of difethialone is such that the ratio of this amount to the (total) amount of said hetero-stereoisomer is greater than 50%.

Advantageously and according to the invention, the composition comprises an amount of the dextrorotatory enantiomer of said hetero-stereoisomer of difethialone such that the ratio of this amount to the (total) amount of said hetero-stereoisomer of difethialone in the composition is greater than 50%, especially greater than 60%, in particular greater than 70%, more particularly greater than 80%, preferably greater than 90%, more preferentially greater than 95%, particularly preferentially greater than 98%, even more preferentially greater than 99% or about 100%. Advantageously, the composition comprises an amount of the dextrorotatory enantiomer of said hetero-stereoisomer of difethialone such that the ratio of this amount to the amount of said hetero-stereoisomer of difethialone in the composition is greater than 75%, preferably between 85% and 100%, more preferentially between 90% and 98%. Advantageously, the composition comprises an amount of the dextrorotatory enantiomer of said hetero-stereoisomer of difethialone such that the ratio of this amount to the amount of said hetero-stereoisomer of difethialone in the composition is between 98% and 100%.

The invention thus relates to a composition in which said hetero-stereoisomer of difethialone is predominantly in dextrorotatory enantiomer form.

In a composition according to the invention:
the ratio of the amount of dextrorotatory enantiomer of said hetero-stereoisomer of difethialone to the sum of the amounts of each of the (laevorotatory and dextrorotatory) enantiomers of said hetero-stereoisomer of difethialone is greater than 0.5 (greater than 50%);
the ratio of the concentration of dextrorotatory enantiomer of said hetero-stereoisomer of difethialone to the sum of the concentrations of each of the (laevorotatory and dextrorotatory) enantiomers of said hetero-stereoisomer of difethialone is greater than 0.5 (greater than 50%); and
the proportion of dextrorotatory enantiomer of said hetero-stereoisomer of difethialone in the composition is greater than the proportion of the other (laevorotatory) enantiomer of said hetero-stereoisomer of difethialone.

Advantageously, the composition may also comprise an amount of laevorotatory enantiomer of said hetero-stereoisomer of difethialone such that the ratio of this amount to the amount of said hetero-stereoisomer of difethialone is less than 50%, especially less than 25%, preferentially between 0% and 25%, in particular less than 10%.

Advantageously and according to the invention, the difethialone is predominantly in the dextrorotatory enantiomer form of said hetero-stereoisomer of difethialone. The composition thus comprises an amount of the dextrorotatory enantiomer of said hetero-stereoisomer of difethialone such that the ratio of this amount to the (total) amount of difethialone is greater than the ratios of the amount of each difethialone enantiomer different from the dextrorotatory enantiomer of said hetero-stereoisomer of difethialone to the (total) amount of difethialone.

Advantageously, in a composition according to the invention:
the ratio of the amount of dextrorotatory enantiomer of said hetero-stereoisomer of difethialone to the sum of the amounts of each of the enantiomers of said hetero-stereoisomer of difethialone and of each of the enantiomers of said homo-stereoisomer of difethialone is greater than 0.25 (greater than 25%);
the ratio of the concentration of dextrorotatory enantiomer of said hetero-stereoisomer of difethialone to the sum of the concentrations of each of the enantiomers of said hetero-stereoisomer of difethialone and of each of the enantiomers of said homo-stereoisomer of difethialone is greater than 0.25 (greater than 25%); and
the proportion of dextrorotatory enantiomer of said hetero-stereoisomer of difethialone in the composition is greater than the proportion of each of the enantiomers of said homo-stereoisomer of difethialone and of the other (laevorotatory) enantiomer of said hetero-stereoisomer of difethialone. In a composition according to the invention, the proportion of dextrorotatory enantiomer of said hetero-stereoisomer of difethialone in the composition is more than 25% relative to the total difethialone.

Advantageously and according to the invention, the composition comprises an amount of the dextrorotatory enantiomer of said hetero-stereoisomer of difethialone such that the ratio of this amount to the amount of difethialone in the composition is greater than 25%, especially greater than 50%, in particular greater than 70%, more particularly greater than 80%, preferably greater than 90%, particularly preferentially greater than 95%, more preferentially greater than 98%, even more preferentially greater than 99% or about 100%.

Advantageously, the composition comprises an amount of the dextrorotatory enantiomer of said hetero-stereoisomer of difethialone such that the ratio of this amount to the amount of difethialone in the composition is greater than 70%, preferably between 80% and 100%, more preferentially between 90% and 100%. Advantageously, the composition comprises an amount of the dextrorotatory enantiomer of said hetero-stereoisomer of difethialone such that the ratio of this amount to the amount of difethialone in the composition is between 95% and 99%. Advantageously, the composition comprises an amount of the dextrorotatory enantiomer of said hetero-stereoisomer of difethialone such that the ratio of this amount to the amount of difethialone in the composition is between 98% and 100%. Advantageously, the composition comprises an amount of the dextrorotatory enantiomer of said hetero-stereoisomer of difethialone such that the ratio of this amount to the amount of difethialone in the composition is substantially about 100%.

Advantageously and according to the invention, the composition comprises an amount of the dextrorotatory enantiomer of said hetero-stereoisomer of difethialone such that the ratio of this amount to the amount of difethialone in the composition is greater than 95%.

A composition according to the invention may be substantially free of laevorotatory enantiomer of said hetero-stereoisomer of difethialone, i.e. the laevorotatory enantiomer of said hetero-stereoisomer of difethialone may be present in the composition, but only in trace amount. It may also be substantially free of said homo-stereoisomer of difethialone, i.e. said homo-stereoisomer of difethialone may be present in the composition, but only in trace amount.

Advantageously and according to the invention, the composition is in liquid form and comprises a liquid solvent for difethialone. It may be a solution of difethialone in a solvent for difethialone, with the exclusion of a racemic mixture of said laevorotatory and dextrorotatory enantiomers of said hetero-stereoisomer of difethialone. It may also be a solution comprising difethialone in a solvent for difethialone and in which said hetero-stereoisomer of difethialone is predominantly in dextrorotatory enantiomer form. It may also be a solution comprising difethialone in a solvent for difethialone and in which the difethialone is predominantly in the dextrorotatory enantiomer form of said hetero-stereoisomer of difethialone.

Advantageously and according to the invention, the composition is in solid form. It may also be a solid comprising difethialone, with the exclusion of a racemic mixture of the dextrorotatory and laevorotatory enantiomers of said hetero-stereoisomer of difethialone. It may also be a solid comprising difethialone and in which said hetero-stereoisomer of difethialone is predominantly in dextrorotatory enantiomer form. It may also be a solid comprising difethialone and in which the difethialone is predominantly in the dextrorotatory enantiomer form of said hetero-stereoisomer of difethialone.

The invention thus also relates to a composition comprising the dextrorotatory enantiomer of said hetero-stereoisomer according to the invention, with the exclusion of a racemic mixture of the laevorotatory and dextrorotatory enantiomers of said hetero-stereoisomer of difethialone, the difethialone of said composition being optically active and dextrorotatory. However, it is not excluded for the difethialone of the composition according to the invention, comprising the dextrorotatory enantiomer according to the invention with the exclusion of a racemic mixture of the laevorotatory and dextrorotatory enantiomers of said hetero-stereoisomer of difethialone, to itself be optically active and laevorotatory, or alternatively optically inactive.

The invention also relates to the use of a composition according to the invention for the preparation of a rodenticidal bait for target rodent pests.

The invention also relates to a rodenticidal bait comprising a composition according to the invention, and at least one excipient that is edible for target rodent pests.

A rodenticidal bait according to the invention comprises:
at least one excipient that is edible for target rodent pests,
the dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations, with the exclusion of a racemic mixture of dextrorotatory and laevorotatory enantiomers of said hetero-stereoisomer of difethialone. In a rodenticidal bait according to the invention, the dextrorotatory enantiomer of said hetero-stereoisomer of difethialone and the laevorotatory enantiomer of said hetero-stereoisomer of difethialone are in unequal (different) amounts.

Advantageously, a bait according to the invention comprises an excipient that is edible for target rodent pests and said hetero-stereoisomer of difethialone predominantly in the dextrorotatory enantiomer form of said hetero-stereoisomer of difethialone. Advantageously, a bait according to the invention comprises an excipient that is edible for target rodent pests and difethialone predominantly in the dextrorotatory enantiomer form of said hetero-stereoisomer of difethialone.

The inventors who succeeded in separating the dextrorotatory enantiomer of said hetero-stereoisomer of difethialone and the laevorotatory enantiomer of said hetero-stereoisomer of difethialone and in isolating the dextrorotatory enantiomer of said hetero-stereoisomer of difethialone also discovered that the dextrorotatory enantiomer of said hetero-stereoisomer of difethialone shows persistence in the liver of target rodent pests and rodenticidal efficacy, in particular at a low dose of difethialone, which are higher than the persistence and the rodenticidal efficacy of the laevorotatory enantiomer of said hetero-stereoisomer of difethialone, and also higher than the persistence and rodenticidal efficacy of the two laevorotatory and dextrorotatory enantiomers of said homo-stereoisomer of difethialone.

Thus, the dextrorotatory enantiomer of said hetero-stereoisomer of difethialone according to the invention allows the production of rodenticidal baits whose efficacy is maintained when compared with the baits of the prior art, but with a reduced proportion of rodenticidal active substance. Such rodenticidal baits are thus less toxic to the environment. Such rodenticidal baits may also be used for controlling populations of target rodent pests that are resistant to known rodenticidal treatments.

Advantageously and according to the invention, the bait comprises a mass amount of difethialone such that the ratio (mass proportion) of this mass amount of difethialone to the mass amount of rodenticidal bait is less than 50 ppm, i.e. less than 50 mg of difethialone per kilogram of rodenticidal bait. Advantageously, the mass proportion of difethialone relative to the bait is greater than 1 ppm. Advantageously, the mass proportion of difethialone relative to the amount of bait is between 1 ppm and 50 ppm (1 mg to 50 mg of difethialone per kilogram of bait), especially between 1 ppm and 40 ppm (1 mg to 40 mg of difethialone per kilogram of bait), preferably between 1 ppm and 25 ppm (1 mg to 25 mg of difethialone per kilogram of bait), more preferentially between 2 ppm and 15 ppm (2 mg to 15 mg of difethialone per kilogram of bait), even more preferentially between 5 ppm and 10 ppm (5 mg to 10 mg of difethialone per kilogram of bait). Advantageously and according to the invention, the bait comprises difethialone in a mass proportion of about 3 ppm.

Advantageously and according to the invention, the rodenticidal bait comprises an amount of the dextrorotatory enantiomer of said hetero-stereoisomer of difethialone such that the ratio of this amount to the amount of difethialone is greater than 70%, more particularly greater than 80%, preferably greater than 90%, particularly preferentially greater than 95%, more preferentially greater than 98%, even more preferentially greater than 99% or about 100%, and of difethialone in a mass proportion of less than 50 ppm relative to the bait.

Advantageously and according to the invention, the bait comprises an amount of the dextrorotatory enantiomer of said hetero-stereoisomer of difethialone such that the ratio of this amount to the amount of difethialone is between 95% and 100% (limits inclusive), and of difethialone in a mass proportion of between 2 ppm and 10 ppm relative to the bait.

Advantageously and according to the invention, the excipient that is edible for target rodent pests is chosen to allow consumption of the bait by target rodent pests. Advantageously and according to the invention, each edible excipient is non-lethal to target rodent pests. The edible excipient is not in itself rodenticidal.

Advantageously and according to the invention, the edible excipient comprises at least one food chosen from the group formed from cereal seeds—especially hulled cereal seeds—cereal seed meals, cereal seed flours, cereal seed flakes, cereal bran and non-cereal seeds, for example alfalfa seeds, especially in hulled form, in the form of meal, in the form of flour, or in the form of flakes or bran. The edible excipient may comprise any support that can be consumed by target rodent pests.

Advantageously, the edible excipient comprises at least one food chosen from the group formed from foods of plant origin and foods of animal origin. Advantageously, the edible excipient comprises at least one food chosen to stimulate the appetite of the target rodent pests. In particular, this food is chosen from the group formed from seeds of one or more cereals, hulled seeds of one or more cereals, meals of seeds of one or more cereals, flakes of seeds of one or more cereals, bran of one or more cereals and flour of seeds of one or more cereals. By way of example, the cereals are chosen from the group formed from oat, wheat, barley, corn, soybean and rice.

Advantageously, the food is chosen from the group formed from sweetened foods. For example, they may be foods comprising at least one sugar chosen from the group formed from sucrose, lactose, fructose and glucose. It may be a sugar syrup—for example a sugar syrup obtained by hydrolysis of starch—or a sugar syrup obtained by hydrolysis of sucrose (invert sugar syrup), or a beet sugar syrup, or a maple syrup or a sugarcane syrup, or a syrup obtained from a plant of the Stevia genus.

Advantageously, the food is chosen from the group formed from coconut albumen (copra) flakes and flour. Advantageously, the food is chosen from the group formed from walnuts, hazelnuts and almonds—in grated and/or powder form.

Advantageously, the food is chosen from the group formed from plant fats, plant oils (for example rapeseed oil, soybean fat, sunflower oil, cocoa butter, groundnut oil, groundnut butter, corn oil, palm oil), animal fats and animal oils (butter, lard, fish oil).

Advantageously, the food is chosen from the group formed from proteins of plant origin and proteins of animal origin. By way of example, examples that may be mentioned include powdered milk—especially powdered skimmed milk—eggs—especially powdered eggs—protein hydrolysates of animal origin and protein hydrolysates of plant origin.

Advantageously and according to the invention, the rodenticidal bait is chosen from the group formed from solid baits comprising difethialone and a solid edible excipient. Advantageously, the rodenticidal bait is a solid in divided form, for example in the form of balls or granules. Advantageously, the rodenticidal bait may be a solid in block or paste form that may be consumed by the target rodent pests or a solid material that may be nibbled by the target rodent pests. Advantageously, the solid rodenticidal bait according to the invention may be in the form of a rigid block, a semi-rigid block, a foam, a powder or a gel.

Advantageously, the rodenticidal bait which is in the form of a powder, in the form of a foam or in the form of a gel is suitable for soiling the fur of the target rodent pest(s) and for being ingested by said pest(s) during their grooming.

It may be a solid rodenticidal bait comprising difethialone, with the exclusion of a racemic mixture of the dextrorotatory and laevorotatory enantiomers of said hetero-stereoisomer of difethialone. It may also be a solid rodenticidal bait comprising difethialone and in which said hetero-stereoisomer of difethialone is predominantly in dextrorotatory enantiomer form. It may also be a solid rodenticidal bait comprising difethialone in which the difethialone is predominantly in the dextrorotatory enantiomer form of said hetero-stereoisomer of difethialone.

Advantageously and according to the invention, the rodenticidal bait is chosen from the group formed from liquid baits comprising difethialone and a liquid edible excipient. The rodenticidal bait is then a drink for target rodent pests. It may be a solution of difethialone in a solvent for difethialone, with the exclusion of a racemic mixture of dextrorotatory and laevorotatory enantiomers of said hetero-stereoisomer of difethialone. It may also be a solution of difethialone in a solvent for difethialone and in which said hetero-stereoisomer of difethialone is predominantly in dextrorotatory enantiomer form. It may also be a solution of difethialone in a solvent for difethialone and in which the difethialone is predominantly in the dextrorotatory enantiomer form of said hetero-stereoisomer of difethialone. It may also be a suspension or an emulsion of difethialone in a liquid medium.

The invention thus also relates to a rodenticidal bait in which the difethialone is optically active. However, it is not excluded for the difethialone of the rodenticidal bait according to the invention to be optically inactive.

Advantageously, the rodenticidal bait comprises at least one dye. Such a dye makes it possible in particular to give said rodenticidal bait a colour that is readily detectable and identifiable by a person handling the rodenticidal bait.

Advantageously, the rodenticidal bait comprises at least one preserving agent capable of ensuring its conservation during its storage. Advantageously, the rodenticidal bait comprises at least one bittering compound such as denatonium benzoate, also known as Bitrex®, which is intended to reduce the risks of accidental consumption by non-target organisms.

Advantageously, in one particular variant, the composition and the rodenticidal bait according to the invention exclusively comprise difethialone—in which the hetero-stereoisomer is not a racemic mixture—as rodenticidal substance. In particular, the composition and the rodenticidal bait according to the invention are free of any other anticoagulant substance for rodenticidal use. However, in this variant according to the invention, the composition and the rodenticidal bait may comprise any pest-control substance other than a rodenticide, such as an insecticidal and/or acaricidal substance.

Advantageously, in another particular variant, the composition and the rodenticidal bait according to the invention comprise difethialone with the exclusion of a racemic mixture of laevorotatory and dextrorotatory enantiomers of said hetero-stereoisomer and at least one other substance different from difethialone as rodenticidal substance. This other rodenticidal substance different from difethialone may be another anticoagulant substance—especially of the anti-vitamin K type or not—or another non-anticoagulant rodenticidal substance.

The invention also relates to a process for controlling target rodent pests, in which there is spread an amount of rodenticidal bait comprising:
at least one excipient that is edible for target rodent pests; and
the dextrorotatory enantiomer of the configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations;
   with the exclusion of a racemic mixture of the dextrorotatory and laevorotatory enantiomers of said hetero-stereoisomer of difethialone.

The invention also relates to a process for controlling target rodent pests, in which there is spread an amount of rodenticidal bait according to the invention, said amount of bait being sufficient to be rodenticidal. An amount of rodenticidal bait in which the dextrorotatory enantiomer of said hetero-stereoisomer of difethialone and the laevorotatory enantiomer of said hetero-stereoisomer of difethialone are in unequal amounts is thus spread.

Advantageously, an amount of rodenticidal bait comprising said hetero-stereoisomer of difethialone predominantly in dextrorotatory enantiomer form is spread. Advantageously, an amount of rodenticidal bait comprising difethialone predominantly in dextrorotatory enantiomer form is spread, the amount of dextrorotatory enantiomer of said hetero-stereoisomer of difethialone that is spread being reduced (bait with a low dose of rodenticidal substance), the bait nevertheless having sufficient rodenticidal efficacy.

In such a process, advantageously and according to the invention, said hetero-stereoisomer of difethialone is predominantly in dextrorotatory enantiomer form.

In such a process, advantageously and according to the invention, the difethialone is predominantly in the dextrorotatory enantiomer form of said hetero-stereoisomer of difethialone.

Advantageously and as a variant according to the invention, the following are chosen in combination:
the edible excipient;
a proportion of dextrorotatory enantiomer of said hetero-stereoisomer of difethialone relative to said hetero-stereoisomer of difethialone;
a proportion of dextrorotatory enantiomer of said hetero-stereoisomer of difethialone relative to the difethialone;
a mass proportion of difethialone relative to the rodenticidal bait; and
an amount of spread bait;
   so that target rodent pests consume an amount of difethialone that is sufficient to be lethal to said target rodent pests which consume said bait in the course of a single period of 24 consecutive hours.

A rodenticidal bait according to this variant of the invention is a bait that is mortal in a single intake, or a “one-shot” bait. Advantageously and according to this variant of the invention, the mass proportion of difethialone in the rodenticidal bait is less than 50 ppm, between 1 ppm and 40 ppm, especially between 1 ppm and 25 ppm, preferably between 5 ppm and 25 ppm, more preferentially between 10 ppm and 25 ppm. Advantageously, the mass proportion of difethialone in the rodenticidal bait may be between 10 ppm and 15 ppm.

Advantageously and in another variant according to the invention, the following are chosen in combination:
the edible excipient;
a proportion of dextrorotatory enantiomer of said hetero-stereoisomer of difethialone relative to said hetero-stereoisomer of difethialone;
a proportion of dextrorotatory enantiomer of said hetero-stereoisomer of difethialone relative to the difethialone;
a mass proportion of difethialone relative to the rodenticidal bait; and
an amount of spread bait;
   so that target rodent pests consume an amount of difethialone:
which is non-lethal to target rodent pests, i.e. which is generally non-lethal to target rodent pests which consume said bait over a period of 24 consecutive hours; and
which is sufficient to be lethal to target rodent pests which consume said bait over several 24-hour periods.

Advantageously, said periods are consecutive.

This other variant of the invention is thus also directed towards a process for controlling target rodent pests, in which there is spread an amount of rodenticidal bait that is lethal to target rodent pests durably consuming this rodenticidal bait and non-lethal to non-target rodents or animals accidentally consuming this rodenticidal bait. This is then referred to as a “multi-dose” or “multi feeding” control process. In such a process according to the invention, the consumption of rodenticidal bait by a target rodent pest over a period of 24 hours is generally insufficient to result in the death of said rodent, whereas repeated consumption of rodenticidal bait over at least two consecutive days results in the death of the target rodent pest.

This other variant of the invention is thus directed towards a process for controlling a population of target rodent pests, in which target rodent pests are provided with an amount of rodenticidal bait that is liable to be ingested by the target rodent pests, said amount of rodenticidal bait being sufficient to kill target rodent pests which consume said rodenticidal bait over several days.

Advantageously, in this other variant of a process according to the invention, the amount of rodenticidal bait spread, the mass proportion of difethialone relative to the rodenticidal bait and the proportion of dextrorotatory enantiomer of said hetero-stereoisomer of difethialone relative to the difethialone are adapted so that the consumption of the rodenticidal bait is lethal to target rodent pests which daily consume bait over at least two 24-hour periods, especially from 3 to 7 periods, said periods being consecutive.

Advantageously, in this other variant of a process according to the invention, since the proportion of dextrorotatory enantiomer of said hetero-stereoisomer of difethialone is greater than 95%—especially about 100%—relative to the difethialone, the mass proportion of difethialone relative to the rodenticidal bait is between 2 ppm and 15 ppm, especially about 10 ppm. In a process according to the invention, an amount of rodenticidal bait that is sufficient to satisfy their daily appetite is provided to target rodent pests, said rodenticidal bait comprising a major proportion of dextrorotatory enantiomer of said hetero-stereoisomer of difethialone.

In a process according to the invention, the amount of rodenticidal bait spread, the proportion of the dextrorotatory enantiomer of said hetero-stereoisomer of difethialone relative to the difethialone and the mass proportion of difethialone relative to the rodenticidal bait are adapted so as to allow consumption of rodenticidal bait for several days by target rodent pests, while at the same time limiting:
the risks of primary intoxication of non-target mammals and birds which are liable to consume such a rodenticidal bait only occasionally and accidentally;
the risks of secondary intoxication, for example of predators of target rodents, which are liable to consume target rodents—dead or live—that have ingested an amount of said bait.

Advantageously and according to the invention, the amount of bait spread, the mass proportion of difethialone relative to the bait and the proportion of dextrorotatory enantiomer of said hetero-stereoisomer of difethialone are adapted to limit the amount of hetero-stereoisomer of difethialone in the liver of rodents which have died by consuming said bait.

The invention also relates to a chromatographic process for obtaining a dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group of said hetero-stereoisomer have different absolute configurations, in which process:
a high-pressure liquid chromatography column of dimensions 150×2 mm, and comprising a chiral stationary phase constituted of particles of tris(4-methylbenzoate) cellulose, said particles having a mean size of 3 μm and having a mean pore size of 1000 Å, is chosen;
a mixture formed from acetonitrile (A) and water comprising 0.1% by volume of formic acid (B), with an A/B volume ratio of 80/20 and with a flow rate of the liquid mobile phase in the chromatography column of 0.25 mL/minute, is chosen as liquid mobile phase;
separation of the configurational stereoisomers of difethialone is performed at room temperature, during which:
a liquid composition comprising said dextrorotatory enantiomer of said hetero-stereoisomer of difethialone is introduced into the top of the chromatography column; and then
the liquid composition is entrained with the mobile phase in the chromatography column under conditions suitable for separating the configurational stereoisomers of difethialone; and
a fraction of the mobile phase comprising said dextrorotatory enantiomer of said hetero-stereoisomer of difethialone is collected with a retention time t₃ having a value such that t₁<t₂<t₃<t₄; t₁, t₂ and t₄ representing the retention times of each of the configurational stereoisomers of difethialone different from the dextrorotatory enantiomer of said hetero-stereoisomer of difethialone, separately from a laevorotatory enantiomer of said hetero-stereoisomer of difethialone with a retention time t₂ and from the laevorotatory and dextrorotatory enantiomers of a configurational stereoisomer of difethialone, named homo-stereoisomer, in which carbons 1 and 3 of the 1,2,3,4-tetrahydronaphthalene group of said homo-stereoisomer have the same absolute configuration, and of retention times t₁ and t₄; and then
the liquid mobile phase of said fraction is removed so as to obtain said dextrorotatory enantiomer of said hetero-stereoisomer of difethialone.

The invention also relates to said dextrorotatory enantiomer of said hetero-stereoisomer of difethialone obtained via a process according to the invention.

The invention also relates to a configurational stereoisomer of difethialone, to a process for obtaining such a configurational stereoisomer of difethialone, to a composition and a rodenticidal bait comprising such a configurational stereoisomer and to a process for controlling target rodent pests, which are characterized in combination by all or some of the characteristics mentioned hereinabove or hereinbelow.

Other aims, characteristics and advantages of the invention will emerge on reading the following description and the examples, which are given for purely non-limiting purposes and which refer to the attached figures, in which:

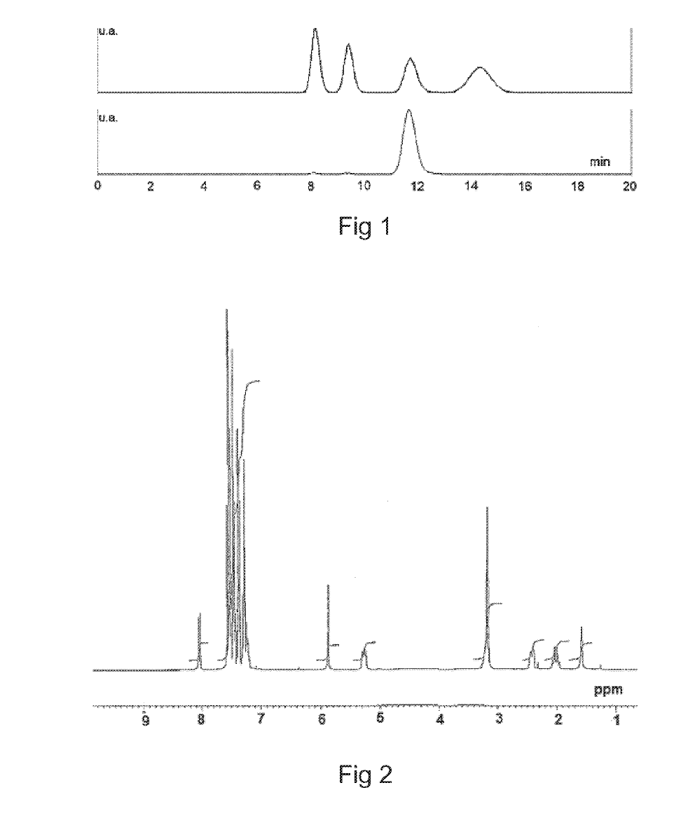

FIG. 1 is a chromatogram of separation and a chromatogram of analysis by high-pressure liquid chromatography on a chiral column of the dextrorotatory enantiomer of said hetero-stereoisomer of difethialone;

FIG. 2 is a proton NMR spectrum at 300 MHz of said hetero-stereoisomer of difethialone;

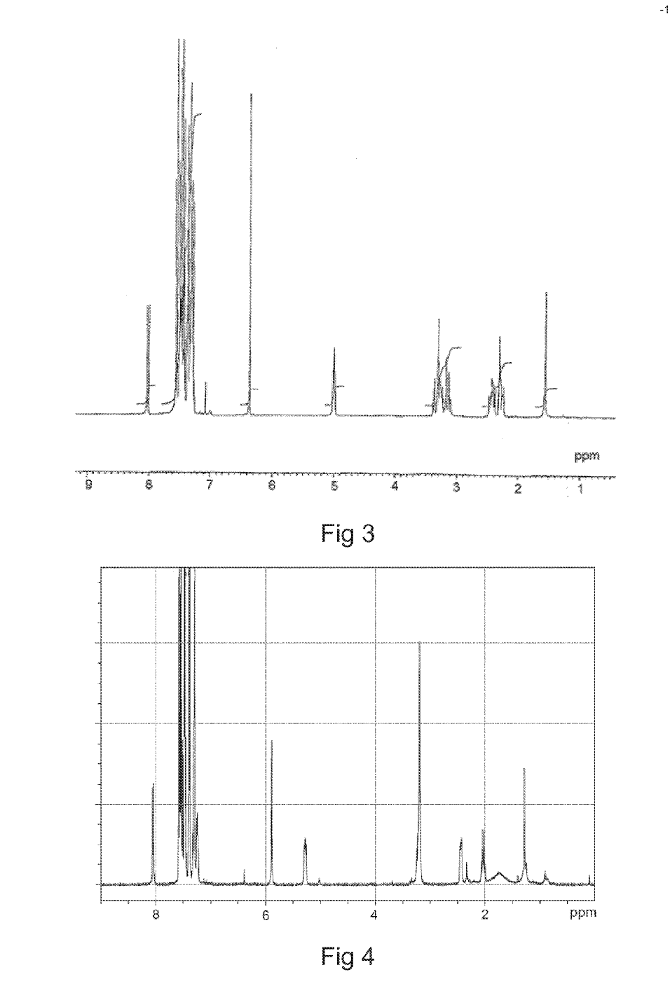

FIG. 3 is a proton NMR spectrum at 300 MHz of said homo-stereoisomer of difethialone;

FIG. 4 is a proton NMR spectrum at 500 MHz of the dextrorotatory enantiomer of said hetero-stereoisomer of difethialone;

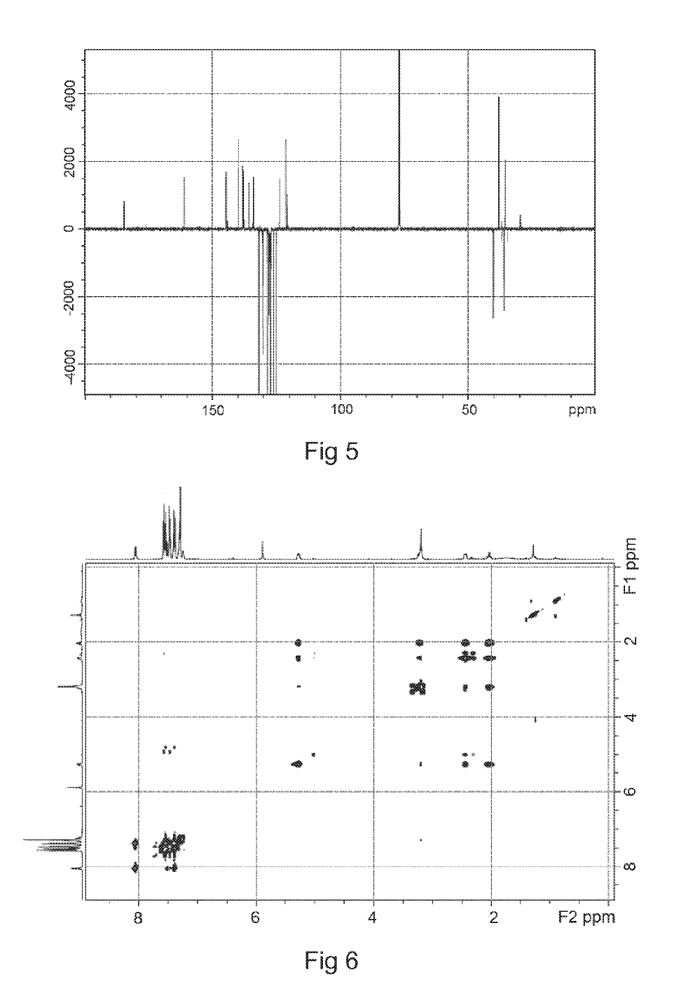

FIG. 5 is a ¹³C carbon NMR spectrum at 500 MHz of the dextrorotatory enantiomer of said hetero-stereoisomer of difethialone;

FIG. 6 is an analysis by proton NMR (¹H-NMR) correlation spectroscopy at 500 MHz of the dextrorotatory enantiomer of said hetero-stereoisomer of difethialone; and

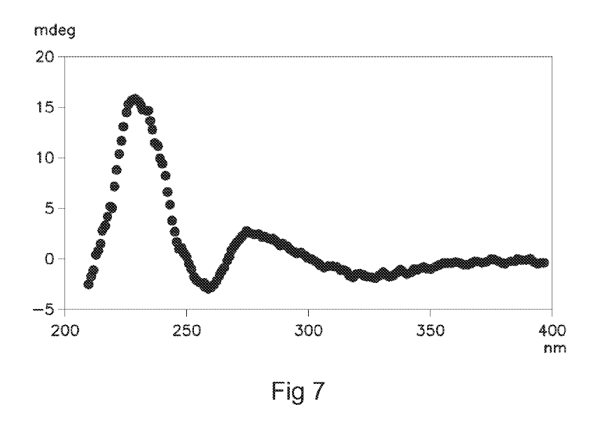

FIG. 7 is a circular dichroism spectrum of the dextrorotatory enantiomer of said hetero-stereoisomer of difethialone.

### A. PURIFICATION OF THE DEXTROROTATORY ENANTIOMER OF SAID HETERO-STEREOISOMER OF DIFETHIALONE

A.1. Identification of Said Hetero-Stereoisomer of Difethialone

Said hetero-stereoisomer of difethialone is identified by proton magnetic resonance (¹H-NMR) spectroscopy. Said hetero-stereoisomer of difethialone dissolved in CDCl₃ has a signal at a chemical shift (δ) of between 5.2 ppm and 5.4 ppm (about 5.3 ppm) and corresponding to the proton borne by carbon 1 of the 1,2,3,4-tetrahydronaphthalene group of difethialone as illustrated in FIG. 2.

Said hetero-stereoisomer is distinguished from said homo-stereoisomer of difethialone dissolved in CDCl₃, which has (FIG. 3) a multiplet at a chemical shift (δ) of between 4.9 ppm and 5.1 ppm and corresponding to the proton borne by carbon 1 of the 1,2,3,4-tetrahydronaphthalene group of difethialone.

A.2. Separation of the Laevorotatory and Dextrorotatory Enantiomers of Said Hetero-Stereoisomer of Difethialone by High-Pressure Liquid Chromatography on a Chiral Column

The inventors solved the complex and hitherto unresolved problem of separating the configurational isomers of difethialone and in particular the laevorotatory and dextrorotatory enantiomers of said hetero-stereoisomer of difethialone. They succeeded in separating the enantiomers of said hetero-stereoisomer of difethialone and of said homo-stereoisomer of difethialone and in isolating the dextrorotatory enantiomer of said hetero-stereoisomer of difethialone by high-pressure (high-performance) liquid chromatography on a LUX® Cellulose-3 chiral column (Phenomenex, Le Pecq, France) of dimensions 150×2 mm and comprising a chiral stationary phase constituted of porous particles of tris(4-methylbenzoate) cellulose, with a particle size of 3 μm and a porosity of 1000 Å. The mobile phase used is an eluent formed from a mixture of acetonitrile (A) and water comprising formic acid in a volume proportion of 0.1% in the water (B) with an A/B volume ratio of 80/20. The flow rate of the mobile phase in the column is 0.25 mL/minute and the separation is performed at a temperature of 23.2° C. The solution containing the sample to be analysed is at a concentration of 1 μg of difethialone per millilitre in acetonitrile and is filtered through a regenerated cellulose membrane with a cut-off threshold of 0.2 μm. The volume of solution containing the sample to be analysed injected onto the column is 1 μL.

In a process for separating the enantiomers of said hetero-stereoisomer of difethialone, it is possible to detect said enantiomers leaving the high-pressure liquid chromatography column by tandem mass spectrometry (MS/MS) in negative electrospray ionization mode (ESI: ElectroSpray Ionization). The temperature of the nebulizer gas is 350° C. and its flow rate is 8 L/minute. The pressure of the nebulizer gas is brought to 2700 hPa. In particular, the MRM (“Multiple Reaction Monitoring”) transitions m/z 537.1→151.0 and m/z 537.1→78.9, corresponding to the difethialone signals, are detected. FIG. 1 represents the chromatograms of difethialone (top) and of the dextrorotatory enantiomer of the isolated hetero-stereoisomer of difethialone (bottom).

Under these experimental conditions, the value of the retention time (t₃) for the dextrorotatory enantiomer of said hetero-stereoisomer of difethialone is about 11.7 minutes as described in FIG. 1, and the value of the retention time (t₂) for the laevorotatory enantiomer of said hetero-stereoisomer of difethialone is about 9.4 minutes, such that the dextrorotatory and laevorotatory enantiomers of said hetero-stereoisomer may be separated by high-pressure liquid chromatography on a chiral column.

Under these same experimental conditions, the value of the retention time (t₄) for said dextrorotatory enantiomer of said homo-stereoisomer according to the invention is about 14.4 minutes, and the value of the retention time (t₁) for the laevorotatory enantiomer of said homo-stereoisomer is about 8.1 minutes, such that the dextrorotatory and laevorotatory enantiomers of said homo-stereoisomer may also be separated by high-pressure liquid chromatography on a chiral column and separated from the dextrorotatory and laevorotatory enantiomers of said hetero-stereoisomer of difethialone.

It is possible under these experimental conditions (stationary phase, mobile phase, temperature) to perform a preparative separation of the laevorotatory and dextrorotatory enantiomers of said hetero-stereoisomer of difethialone by using a similar stationary phase with a particle size of greater than 3 μm, and a chromatography column of larger dimensions, especially a diameter of 20 mm.

### B. STRUCTURAL CHARACTERIZATION

B.1. UV Spectroscopy

The UV spectrum of the dextrorotatory enantiomer of said hetero-stereoisomer of difethialone dissolved in chloroform (CHCl₃) shows two absorbance peaks centred at 238.2 nm and at 259.5 nm.

B.2. Optical Rotation

The inventors characterized the dextrorotatory enantiomer of said hetero-stereoisomer of difethialone in isolated form by means of its optical rotation (also known as the optical activity or circular birefringence), i.e. its ability to deviate the polarization plane of polarized light. Deviation of the polarization plane of polarized light clockwise facing the polarized light beam characterizes a dextrorotatory solution, and deviation of the polarization plane of polarized light anticlockwise facing the polarized light beam characterizes a laevorotatory solution and compound.

The optical rotation of a solution of dextrorotatory enantiomer of said hetero-stereoisomer of difethialone in chloroform (CHCl₃) is measured at a concentration of 6.3 g/L. The optical rotation of this solution is measured by means of a P 2000 digital polarimeter (JASCO, Bouguenais, France) operating with excitatory light with a wavelength of 589 nm. The mean optical rotation a obtained on two series of ten measurements is +0.573°. The specific optical rotation at 25° C. [α]^{25° C.}_{589 nm} for the dextrorotatory enantiomer of said hetero-stereoisomer of difethialone dissolved in chloroform, measured on the sodium D line (589 nm), is +9.1°.

B.3. Circular Dichroism

The circular dichroism spectrum of the dextrorotatory enantiomer of said isolated hetero-stereoisomer of difethialone reflects the difference in absorbance (ΔA=A_{L}−A_{R}) of the two waves of left circular polarization (LCP) of intensity A_{L} and of right circular polarization (RCP) of intensity A_{R}. This makes it possible to distinguish the dextrorotatory and laevorotatory enantiomers of said homo-stereoisomer of difethialone. This difference in absorbance of the two circularly polarized waves is measured in a J-815 circular dichroism spectrometer (JASCO, Bouguenais, France). 2 mL of a solution of dextrorotatory enantiomer of said hetero-stereoisomer of difethialone in methanol (CH₃OH) at a concentration of 0.65 mg/mL are prepared. The solution is transferred into a quartz spectrophotometer cuvette. The circular dichroism spectrum of the solution is measured at 25° C. between 200 nm and 400 nm. The circular dichroism spectrum of the dextrorotatory enantiomer of said hetero-stereoisomer of difethialone measured under these conditions is shown in FIG. 7. The circular dichroism value is positive between the wavelengths of 215 nm and 250 nm and between 270 nm and 300 nm.

B.4. Nuclear Magnetic Resonance

FIG. 4 is a proton NMR spectrum at 500 MHz of the dextrorotatory enantiomer of said hetero-stereoisomer of difethialone dissolved at a concentration of 40 mg/mL in CDCl₃, having a signal at a chemical shift (δ) of about 5.3 ppm and corresponding to the proton borne by carbon 1 of the 1,2,3,4-tetrahydronaphthalene group of difethialone.

FIG. 5 is a ¹³C NMR spectrum of the dextrorotatory enantiomer of said hetero-stereoisomer of difethialone dissolved at a concentration of 40 mg/mL in CDCl₃, acquired on a Bruker Avance III HD spectrometer (500 MHz) equipped with a Prodigy motorized multi-core direct cryoprobe. It allows identification of the 31 carbon atoms of difethialone. The ¹³C-NMR spectrum of the dextrorotatory enantiomer of said hetero-stereoisomer of difethialone is not distinguished from the ¹³C-NMR spectrum of the laevorotatory enantiomer of said hetero-stereoisomer of difethialone. However, said hetero-stereoisomer of difethialone has signals between 40 ppm and 40.5 ppm and between 38.0 ppm and 38.5 ppm which are characteristic of said hetero-stereoisomer of difethialone and different from said homo-stereoisomer of difethialone.

FIG. 6 is a two-dimensional proton NMR (2D ¹H-NMR) spectrum obtained by correlation spectroscopy of the dextrorotatory enantiomer of said hetero-stereoisomer of difethialone dissolved in CDCl₃ at a concentration of 40 mg/mL acquired on a Bruker Avance III HD spectrometer (500 MHz) equipped with a Prodigy motorized multi-core direct cryoprobe. It allows identification of the coupling of the proton borne by carbon 1 of the 1,2,3,4-tetrahydronaphthalene group of said homo-stereoisomer of difethialone (5.3 ppm) with the protons borne by carbon 2 of the 1,2,3,4-tetrahydronaphthalene group at 2.0 ppm and 2.4 ppm.

### C. EXTRACTION OF DIFETHIALONE FROM THE LIVER OF RATS TREATED WITH DIFETHIALONE FOR THE PURPOSE OF ANALYSIS OF THE VARIOUS CONFIGURATIONAL STEREOISOMERS OF DIFETHIALONE

C.1. Homogenization of the Liver Sample

About 0.525 g (±0.025 g) of rat liver is weighed out accurately and placed in a 50 mL polypropylene tube. 10 mL of acetone are added and the suspension is homogenized using an Ultra-Turrax® homogenizer/disperser for a time of about 30 seconds. The homogenizer/disperser shaft is rinsed with hot water and then twice with 20 mL of acetone in a polypropylene tube. The homogenate is centrifuged for 5 minutes at a centrifugation speed of 3000 rpm (revolutions per minute). The supernatant is collected and transferred into a test tube. The sample is subjected to evaporation under a stream of nitrogen (N₂) at a temperature of 40° C. so as to form a dry extract.

C.2. Lipid Removal

1 mL of acetonitrile is added to the tube containing the dry extract so as to dissolve it. The acetonitrile solution is washed twice successively with 1 mL of hexane. The lipid-free extract is dried under a stream of nitrogen (N₂) at a temperature of 40° C. and is then taken up in 0.5 mL of methanol and dissolved by vortex stirring. 0.5 mL of ultra-pure (Milli-Q) water is then added. The sample is vortex-homogenized.

C.3. Solid-Phase Extraction (SPE) of Difethialone

1 mL of dichloromethane (CH₂Cl₂), then 1 mL of methanol (CH₃OH), then 1 mL of ultra-pure (Milli-Q) water are passed through an Oasis HLB 1 cc cartridge (WAT094225, Waters). The lipid-free liver extract (1 mL MeOH/Milli-Q H₂O) containing difethialone is then loaded onto the top of the preconditioned cartridge. The liver extract penetrates through the cartridge by gravity on contact with the solid phase of the cartridge. 1 mL of washing solution formed from methanol (CH₃OH) and ultra-pure water (H₂O) in a 90/10 volume proportion is loaded onto the top of the cartridge. The cartridge is dried by suction under vacuum connected to the bottom of the cartridge. 1 mL of eluting solution formed from dichloromethane (CH₂Cl₂) and methanol (CH₃OH) in a 90/10 volume proportion is then loaded onto the top of the cartridge and an eluate comprising difethialone is collected at the bottom of the cartridge. The solvent of the eluate is evaporated off under a stream of nitrogen (N₂) at a temperature of 40° C.

C.4. Analysis

The solution containing difethialone is analysed by high-pressure liquid chromatography on a LUX® Cellulose-3 chiral column (Phenomenex, Le Pecq, France) (150×2 mm, particle size of 3 μm) as described in point A2) above.

### D. RODENTICIDAL BAIT COMPRISING A PROPORTION OF 3.7 PPM OF DIFETHIALONE

A pasty rodenticidal bait according to the invention is prepared by dispersing an amount of dextrorotatory enantiomer of said hetero-stereoisomer of difethialone in an edible excipient comprising vegetable fat and cereal flour. The measured proportion of difethialone relative to the rodenticidal bait is 3.7 ppm (3.57 mg of dextrorotatory enantiomer of said hetero-stereoisomer per kilogram of bait) and the proportion of dextrorotatory enantiomer of said hetero-stereoisomer relative to the difethialone is 96.5%.

On D0, ten coumaphen-sensitive Sprague-Dawley rats (five male and five female SD rats) are placed in individual cages with a rodenticide-free reference feed. On D3, each rat is weighed, and 50 g of rodenticidal bait as described above are then provided to each rat. This provision of 50 g of rodenticidal bait is renewed daily. The bait consumed by the rats is made up to 50 g of bait on D4, D5 and D6. Starting from D7, the residual rodenticidal baits are removed and rodenticide-free feed is provided to all the rats. The rats are monitored for 3 weeks.

The mean amounts of bait consumed daily by a rat on D4, D5, D6 and D7 expressed in grams per day are given in table 1 below.

TABLE 1

| Bait consumed, g | Mean | Standard deviation |
|---|---|---|
| D 4 | 16.6 | 3.8 |
| D 5 | 15.4 | 5.8 |
| D 6 | 14.3 | 6.2 |
| D 7 | 8.6 | 2.8 |

It should be noted that no rat consumed a daily amount of bait of less than 1 g/day. All the rats (100%) die between D9 and D13. The mortality observed is 100%.

The hepatic content of the dextrorotatory enantiomer of said hetero-stereoisomer of difethialone in each of the rats which die between D9 and D13 is measured by high-pressure liquid chromatography on a chiral column. The measured values, expressed as micrograms of dextrorotatory enantiomer of said hetero-stereoisomer (“DFN-Hetero-dextro”) per gram of liver, are given in table 2 below.

TABLE 2

| Hepatic content, μg/g | “DFN-Hetero-dextro” | Total difethialone |
|---|---|---|
| Mean | 5.365 | 5.372 |
| Standard deviation | 0.828 | 0.842 |

The bait containing a 3.7 ppm dose of dextrorotatory enantiomer of said hetero-stereoisomer of difethialone affords a mortality rate of 100% while minimizing the risks of primary and secondary intoxication.

### E. RODENTICIDAL BAIT COMPRISING A PROPORTION OF 10.6 PPM OF DIFETHIALONE

A pasty rodenticidal bait according to the invention is prepared as described for the above 3.7 ppm bait. The measured proportion of difethialone in the bait is 10.6 ppm (10.2 mg of dextrorotatory enantiomer of said hetero-stereoisomer per kilogram of bait) and the proportion of dextrorotatory enantiomer of said hetero-stereoisomer relative to the difethialone is 96.5%. Bait is dispensed as described above with the 3.7 ppm bait.

The mean amounts of bait consumed daily by a rat on D4, D5, D6 and D7 expressed in grams per day are given in table 3 below.

TABLE 3

| Bait consumed, g | Mean | Standard deviation |
|---|---|---|
| D 4 | 14.9 | 2.3 |
| D 5 | 15.8 | 2.8 |
| D 6 | 13.2 | 1.7 |
| D 7 | 7.4 | 4.6 |

It should be noted that no rat consumed a daily amount of bait of less than 1 g/day. All the rats (100%) die between D5 and D8. The mortality rate obtained with this bait is 100%.

The hepatic content of the dextrorotatory enantiomer of said hetero-stereoisomer of difethialone in each of the rats which die between D5 and D8 is measured by high-pressure liquid chromatography analysis on a chiral column. The values, expressed as micrograms of dextrorotatory enantiomer of said hetero-stereoisomer (“DFN-Hetero-dextro”) per gram of liver, are given in table 4 below.

TABLE 4

| Hepatic content, μg/g | “DFN-Hetero-dextro” | Total difethialone |
|---|---|---|
| Mean | 16.049 | 15.113 |
| Standard deviation | 9.872 | 7.318 |

The bait containing a 10.6 ppm dose of difethialone allows a mortality rate of 100% to be obtained while at the same time minimizing the risks of primary and secondary intoxication.

It goes without saying that the invention may be the subject of numerous implementation variants and applications. In particular, a composition, a rodenticidal bait and a process for controlling target rodent pests are subject to an infinite number of variants both in the formulation of the bait and in the embodiments of the process.

1. Dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations.

2. Composition comprising the dextrorotatory enantiomer according to claim 1, with the exclusion of a racemic mixture of dextrorotatory and laevorotatory enantiomers of said hetero-stereoisomer of difethialone.

3. Composition according to claim 2, wherein said hetero-stereoisomer is predominantly in dextrorotatory enantiomer form.

4. Composition according to claim 2, wherein the difethialone is predominantly in the dextrorotatory enantiomer form of said hetero-stereoisomer of difethialone.

5. Composition according to claim 2, further comprising an amount of the dextrorotatory enantiomer of said hetero-stereoisomer of difethialone such that the ratio of this amount to the amount of difethialone is greater than 25%.

6. Composition according to claim 2, further comprising an amount of the dextrorotatory enantiomer of said hetero-stereoisomer of difethialone such that the ratio of this amount to the amount of difethialone is greater than 95%.

7. Rodenticidal bait comprising a composition according to claim 2 and at least one excipient that is edible for target rodent pests.

8. Bait according to claim 7, wherein the edible excipient comprises at least one food chosen from the group formed from cereal seeds, cereal seed meals, cereal seed flours, cereal seed flakes, cereal bran and non-cereal seeds.

9. Bait according to claim 7, further comprising a mass amount of difethialone such that the ratio of this mass amount of difethialone to the mass amount of rodenticidal bait is less than 50 ppm.

10. Process for controlling target rodent pests, in which there is spread an amount of bait comprising:
at least one excipient that is edible for target rodent pests; and
the dextrorotatory enantiomer of the configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group have different absolute configurations;
with the exclusion of a racemic mixture of the dextrorotatory and laevorotatory enantiomers of said hetero-stereoisomer of difethialone.

11. Process according to claim 10, wherein said hetero-stereoisomer of difethialone is predominantly in dextrorotatory enantiomer form.

12. Process according to claim 10, wherein the difethialone is predominantly in the dextrorotatory enantiomer form of said hetero-stereoisomer of difethialone.

13. Process according to claim 10, wherein the following are chosen in combination:
the edible excipient;
a proportion of dextrorotatory enantiomer of said hetero-stereoisomer of difethialone relative to said hetero-stereoisomer of difethialone;
a proportion of dextrorotatory enantiomer of said hetero-stereoisomer of difethialone relative to the difethialone;
a mass proportion of difethialone relative to the rodenticidal bait; and
an amount of spread bait;
so that target rodent pests consume an amount of difethialone that is sufficient to be lethal to said target rodent pests which consume said bait in the course of a single period of 24 consecutive hours.

14. Process according to claim 10, wherein the following are chosen in combination:
the edible excipient;
a proportion of dextrorotatory enantiomer of said hetero-stereoisomer of difethialone relative to said hetero-stereoisomer of difethialone;
a proportion of dextrorotatory enantiomer of said hetero-stereoisomer of difethialone relative to the difethialone;
a mass proportion of difethialone relative to the rodenticidal bait; and
an amount of spread bait;
so that target rodent pests consume an amount of difethialone:
which is non-lethal to target rodent pests which consume said bait over a period of 24 consecutive hours; and
which is sufficient to be lethal to target rodent pests which consume said bait over several 24-hour periods, said periods being consecutive.

15. Chromatographic process for obtaining a dextrorotatory enantiomer of a configurational stereoisomer of difethialone, named hetero-stereoisomer, the formula of which is 3-(4′-bromobiphenyl-4-yl)-1-(4-hydroxythiocoumarin-3-yl)-1,2,3,4-tetrahydronaphthalene, in which carbons 1 and 3 of the 1,2,3,4-tetrahydronaphthalene group of said hetero-stereoisomer have different absolute configurations, in which process:
a high-pressure liquid chromatography column of dimensions 150×2 mm, and comprising a chiral stationary phase constituted of particles of tris(4-methylbenzoate) cellulose, said particles having a mean size of 3 μm and having a mean pore size of 1000 Å, is chosen;
a mixture formed from acetonitrile (A) and water comprising 0.1% by volume of formic acid (B), with an A/B volume ratio of 80/20 and with a flow rate of the liquid mobile phase in the chromatography column of 0.25 mL/minute, is chosen as liquid mobile phase;
separation of the configurational stereoisomers of difethialone is performed at room temperature, during which:
a liquid composition comprising said dextrorotatory enantiomer of said hetero-stereoisomer of difethialone is introduced into the top of the chromatography column; and then
a fraction of the mobile phase comprising said dextrorotatory enantiomer of said hetero-stereoisomer of difethialone is collected with a retention time t3 having a value such that t1<t2<t3<t4; t1, t2 and t4 representing the retention times of each of the configurational stereoisomers of difethialone different from the dextrorotatory enantiomer of said hetero-stereoisomer of difethialone, separately from a laevorotatory enantiomer of said hetero-stereoisomer of difethialone with a retention time t2 and separately from the laevorotatory and dextrorotatory enantiomers of a configurational stereoisomer of difethialone, named homo-stereoisomer, in which carbons 1 and 3 of the 1,2,3,4-tetrahydronaphthalene group of said homo-stereoisomer have the same absolute configuration, and of retention times t1 and t4; and then
the liquid mobile phase of said fraction is removed so as to obtain said dextrorotatory enantiomer of said hetero-stereoisomer of difethialone.

16. Composition according to claim 3, wherein the difethialone is predominantly in the dextrorotatory enantiomer form of said hetero-stereoisomer of difethialone.

17. Composition according to claim 3, further comprising an amount of the dextrorotatory enantiomer of said hetero-stereoisomer of difethialone such that the ratio of this amount to the amount of difethialone is greater than 25%.

18. Composition according to claim 4, further comprising an amount of the dextrorotatory enantiomer of said hetero-stereoisomer of difethialone such that the ratio of this amount to the amount of difethialone is greater than 25%.

19. Composition according to claim 3, further comprising an amount of the dextrorotatory enantiomer of said hetero-stereoisomer of difethialone such that the ratio of this amount to the amount of difethialone is greater than 95%.

20. Composition according to claim 4, further comprising an amount of the dextrorotatory enantiomer of said hetero-stereoisomer of difethialone such that the ratio of this amount to the amount of difethialone is greater than 95%.
